(19) United States
(12) Patent Application Publication
(10) Pub. No.: US 2004/0249402 A1
(43) Pub. Date: Dec. 9, 2004

(54) Low profile tongue scraper

(76) Inventor: Edward F. Von Bergen, Lake Geneva, WI (US)

Correspondence Address: Donald J. Ersler, 725 Garvens Avenue, Brookfield, WI, 53005, US

(21) Appl. No.: 10/455,663

(22) Filed: Jun. 5, 2003

(51) Int. Cl. A61F 9/00

(52) U.S. Cl. 606/161

(57) ABSTRACT

A low profile tongue scraper includes a handle and a scraping element extending from an end of the handle. A portion of the length of the handle is preferably curved to provide improved access to a bottom portion of the tongue and to reduce the tendency of the user to experience a gag reflex. The scraping element is rigid to facilitate removable of undesirable build-up on the tongue. The scraping element preferably extends from the handle at an acute angle relative to the handle. A plurality of tooth brush bristles are preferably formed on the other end of the handle.

### BACKGROUND OF THE INVENTION

1. Field of the Invention

The present invention relates generally to oral hygiene and more specially to a low profile tongue scraper, which allows the lower portion of the tongue to be cleaned.

2. Discussion of the Prior Art

The prior art provides numerous tongue scrapers. Some of these prior art tongue scrapers include U.S. Pat. No. 5,810,856 to Tveras and Pat. No. 5,827,308 to Thakur et al. The Tveras patent discloses a wiping element for an oral hygiene device, window wiper, or the like. The Tveras patent includes a device for wiping or scraping the human tongue with one or more wiping elements. However, each wiping element is flexible. Further, the width and height of the wiping elements limit how far the Tveras device may be inserted down the throat for scraping the lower portion of the tongue. The Thakur et al. patent discloses a tongue scraping apparatus. The Thakur et al. patent includes a tongue scraping apparatus for use in cleaning the upper surface of a user's tongue. However, the tongue scraping apparatus is not suitable for scraping the lower portion of the tongue.

Accordingly, there is a clearly felt need in the art for a low profile tongue scraper, which is suitable for scraping the lower portion of the tongue, while reducing the probably of inducing a gag reflex.

### SUMMARY OF THE INVENTION

The present invention provides a low profile tongue scraper, which enables the lower portion of the tongue to be cleaned. A low profile tongue scraper includes a handle and a scraping element extending from an end of the handle. The handle is preferably curved to provide improved access to a bottom portion of the tongue and to reduce the tendency of the user to experience a gag reflex. The scraping element is rigid to facilitate removal of undesirable build-up on the tongue. The scraping element preferably extends from the handle at an acute angle relative to the handle. A plurality of tooth brush bristles are preferably formed on the other end of the handle.

Accordingly, it is an object of the present invention to provide a low profile tongue scraper suitable for scraping the lower portion of the tongue that reduces the probability of inducing a gag reflex.

These and additional objects, advantages, features and benefits of the present invention will become apparent from the following specification.

### BRIEF DESCRIPTION OF THE DRAWINGS

FIG. 1 is a perspective view of a low profile tongue scraper in accordance with the present invention.

FIG. 2 is a top view of a low profile tongue scraper in accordance with the present invention.

FIG. 3 is a side view of a low profile tongue scraper in accordance with the present invention.

FIG. 4 is an enlarged side view of a scraper element of a low profile tongue scraper in accordance with the present invention.

### DETAILED DESCRIPTION OF THE PREFERRED EMBODIMENTS

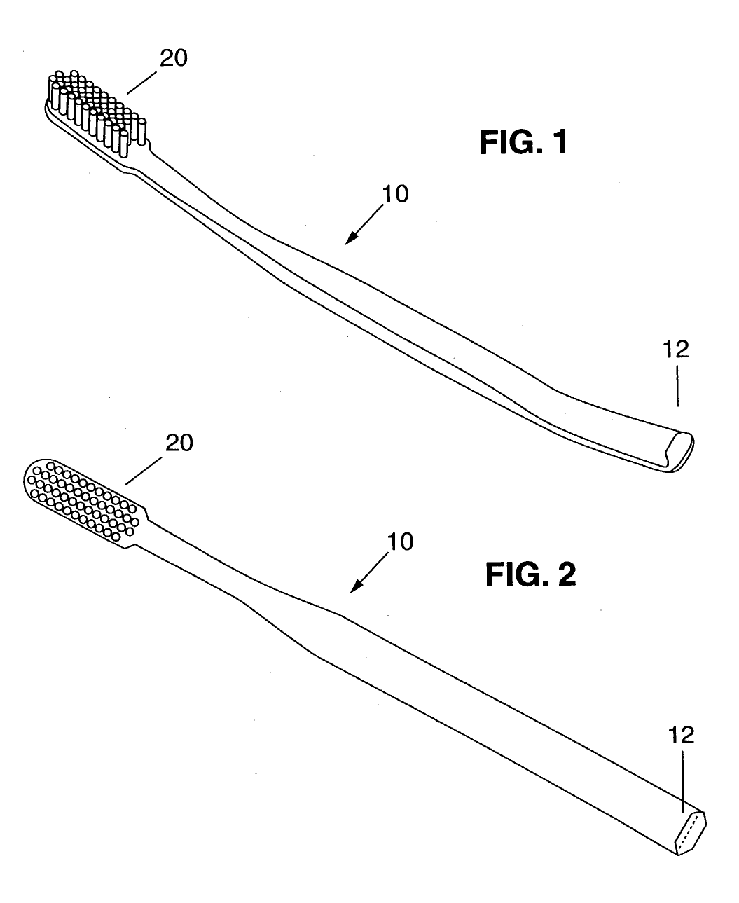

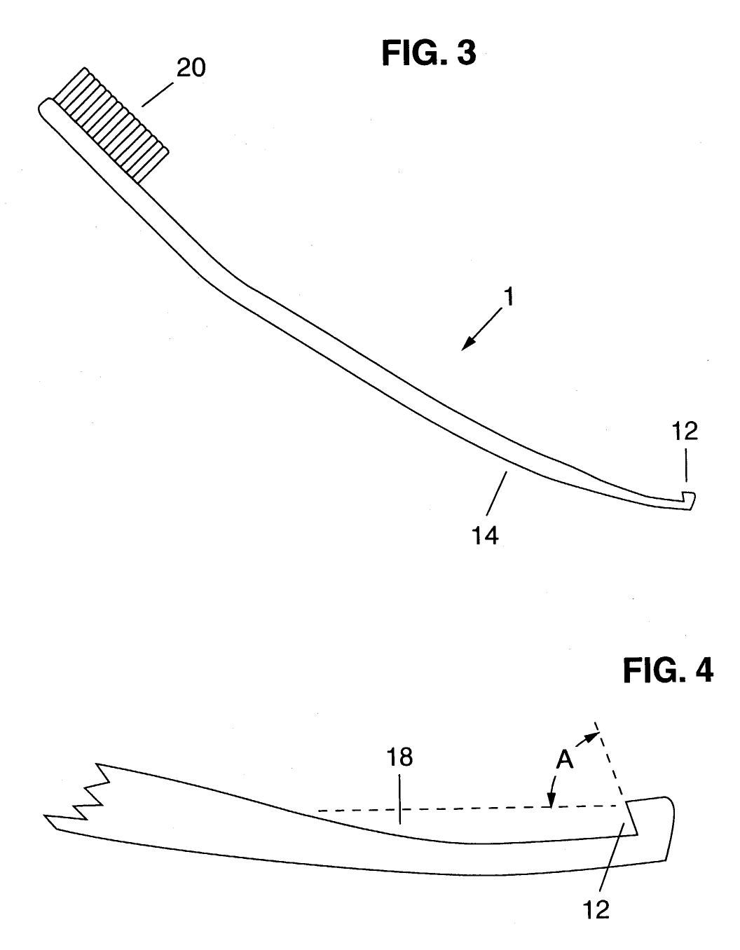

With reference now to the drawings, and particularly to FIG. 1, there is shown a perspective view of a low profile tongue scraper 1. With reference to FIGS. 2-3, the low profile tongue scraper 1 includes a handle 10 and a scraping element 12 extending from one end of the handle 12. A curvature 14 is preferably formed on a portion of a length of the handle 10, starting adjacent the one end to provide improved access to the bottom portion of the tongue. However, the handle 10 should not be limited to a specific shape, but should include any suitable shape. With reference to FIG. 4, the scraping element 12 is preferably formed by removing material from the handle 10 to form a scraper clearance 18. The handle 10 is preferably fabricated from a rigid plastic material, such that the scraping element 12 is rigid to facilitate removable of undesirable build-up on the tongue.

The scraping element 12 preferably extends from the handle 12 at an acute angle “A” relative to the handle 12. The angle “A” has a preferably range of between 45-90 degrees, but other angle values may also be used. Preferably, the scraping element 12 does not extend beyond the profile of the handle 10. The scraping element 12 not extending beyond the profile of the handle 10, allows the handle 10 to be inserted into a tooth brush holder receptacle hole. Scraping elements on prior art tooth brushes typically exceed the profile or thickness of the handle and cannot be inserted into a tooth brush holder receptacle hole. A plurality of tooth brush bristles 20 are preferably formed on the other end of the handle 10.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

I claim:

1. A low profile tongue scraper comprising:
a handle having a scraping element formed on one end thereof, said scraping element extending from said handle at an acute angle relative to said handle, said handle having a curvature formed on a portion of a length thereof.

2. The low profile tongue scraper of claim 1, further comprising:
a scraper clearance being formed in said handle to create said scraping element.

3. The low profile tongue scraper of claim 1, further comprising:
a plurality of tooth brush bristles being formed on the other end of said handle.

4. The low profile tongue scraper of claim 1, further comprising:
said scraping element not extending beyond a profile of said handle.

5. A low profile tongue scraper comprising:
a handle having a scraping element formed on one end thereof, a scraper clearance being formed in said handle to create said scraping element, said scraping element extending from said handle at an acute angle relative to said handle, said handle having a curvature formed on a portion of a length thereof, said curvature starting adjacent to said scraping element.

6. The low profile tongue scraper of claim 5, further comprising:
a plurality of tooth brush bristles being formed on the other end of said handle.

7. The low profile tongue scraper of claim 5, further comprising:
said scraping element not extending beyond a profile of said handle.

8. A low profile tongue scraper comprising:
a handle having a scraping element formed on one end thereof, a scraper clearance being formed in said handle to create said scraping element, said scraping element extending from said handle at an acute angle relative to said handle, said handle having a curvature formed on a portion of a length thereof, said curvature starting adjacent to said scraping element; and
a plurality of tooth brush bristles being formed on the other end of said handle.

9. The low profile tongue scraper of claim 8, further comprising:
said scraping element not extending beyond a profile of said handle.
